(19) United States
(12) Patent Application Publication
(10) Pub. No.: US 2026/0042718 A1
(43) Pub. Date: Feb. 12, 2026

(54) CATALYST SYSTEM AND PROCESS FOR CHEMICALLY TREATING A CARBON-CONTAINING FEEDSTOCK USING THE CATALYST SYSTEM

(71) Applicant: BRASKEM AMERICA, INC., Philadelphia, PA (US)

(72) Inventors: Vibin Vargheese, Pittsburgh, PA (US); Nichoias Wang, Pittsburgh, PA (US); Matthew Feeney, Pittsburgh, PA (US); Scott Mitchell, Pittsburgh, PA (US)

(21) Appl. No.: 19/294,581

(22) Filed: Aug. 8, 2025

Related U.S. Application Data
(60) Provisional application No. 63/680,787, filed on Aug. 8, 2024.

(51) Int. Cl. C07C 4/08; B01J 21/04; B01J 21/08; B01J 23/883; B01J 27/051; B01J 29/16; B01J 37/02; B01J 37/08; B01J 37/20

(52) U.S. Cl. CPC C07C 4/08; B01J 21/04; B01J 21/08; B01J 23/883; B01J 27/0515; B01J 29/166; B01J 37/0205; B01J 37/0207; B01J 37/0236; B01J 37/088; B01J 37/20; B01J 2229/186

(57) ABSTRACT

This invention relates to a process for chemically treating a carbon-containing feedstock (e.g., a polymer-based feedstock), comprising contacting (e.g., by a hydrocracking reaction) the carbon-containing feedstock and a hydrogen stream in the presence of at least one hydrocracking catalyst to produce an alkane-containing product stream. The hydrocracking catalyst comprises at least one transition metal or transition metal sulfide supported on an oxide-containing support. This invention also relates to an alkane-containing mixture obtained by the process described herein and a system/apparatus for carrying out the process described herein.

### PRIORITY CLAIM

This application claims priority under 35 U.S.C. § 119(e) to U.S. Provisional Application No. 63/680,787, filed Aug. 8, 2024, herein incorporated by reference in its entirety.

### FIELD OF THE INVENTION

This invention generally relates to a catalyst system and process for chemically treating a carbon-containing feedstock using the catalyst system.

### BACKGROUND OF THE INVENTION

Plastic waste is a diverse feedstock which may contain several different types of polymers, fillers, stabilizing agents, and/or contaminants. The inconsistent quality of the feedstock provides a technical challenge in the conversion of plastics to chemicals.

The current technologies in conversion of plastics to chemicals typically employ a multi-stage process. The first stage involves thermal cracking (pyrolysis) of waste plastic to pyrolysis oil and gas, which may be carried out with or without a catalyst. These products, however, have a significant weight fraction of unsaturated hydrocarbons. Thus, a second stage is typically necessary for hydrogenating the unsaturated hydrocarbons to saturated hydrocarbons, under high hydrogen pressure over a hydrogenation catalyst.

However, the conventional process generates large amounts of unsaturated, aromatic, and polyaromatic hydrocarbons, which may not been completely converted to saturated hydrocarbons. Additionally, multi-stage process is cumbersome and requires additional pyrolysis units.

There thus remains a need in the art for a process and system to more efficiently crack the polymer feedstock, while increasing the yield and quality of the recovered liquid hydrocarbon product suitable for further cracking.

### SUMMARY OF THE INVENTION

One aspect of the invention relates to a catalyst system comprising at least one transition metal or transition metal sulfide supported on an oxide-containing support having a low acidity, and an acidic zeolite.

Another aspect of the invention relates to the use of a catalyst system comprising at least one transition metal or transition metal sulfide supported on an oxide-containing support having a low acidity, and an acidic zeolite to produce an alkane-containing product stream.

Another aspect of the invention relates to a process for chemically treating a carbon-containing feedstock, the process comprising contacting the carbon-containing feedstock with a hydrogen-containing stream in the presence of a catalyst system, the catalyst system comprising at least one transition metal or transition metal sulfide supported on an oxide-containing support having a low acidity, and an acidic zeolite to produce an alkane-containing product stream.

Another aspect of the invention relates to a process for depolymerizing a polymer-based feedstock comprising reacting a polymer-based feedstock with a hydrogen-containing stream in the presence of a catalyst system, the catalyst system comprising at least one transition metal or transition metal sulfide supported on an oxide-containing support having a low acidity, and an acidic zeolite, to depolymerize the polymer-based feedstock and form an alkane-containing product stream.

Another aspect of the invention relates to an alkane-containing mixture obtained by the processes for chemically treating a carbon-containing feedstock or for depolymerizing a polymer-based feedstock of the present disclosure. The alkane-containing mixture resulting from such processes may comprise at least 50% by weight of naphtha or naphtha-like product, based on the total weight of the mixture.

Another aspect of the invention relates to a method for selectively converting a polymer-based feedstock to a liquid naphtha or naphtha-like product, comprising reacting a polymer-based feedstock with a hydrogen-containing stream in the presence of a catalyst system comprising at least one transition metal or transition metal sulfide supported on an oxide-containing support having a low acidity, and an acidic zeolite to depolymerize the polymer-based feedstock and form a liquid naphtha or naphtha-like product containing at least 50% by weight of C₄-C₁₂ hydrocarbons (e.g., C₄-C₁₂ alkanes); less than 10% (such as less than 5%, less than 3% or less than 1%) by weight of solid product stream (such as C₂₀₊ hydrocarbons); and optionally less than 10% (such as less than 5%, less than 3% or less than 1%) by weight of diesel, kerosene or kerosene like product (such as C₁₃-C₂₀ hydrocarbons), wherein the reaction temperature ranges from about 300-450° C. and hydrogen pressure ranges from about 5 to 100 bar.

Additional aspects, advantages and features of the invention are set forth in this specification, and in part will become apparent to those skilled in the art on examination of the following or may be learned by practice of the invention. The inventions disclosed in this application are not limited to any particular set of or combination of aspects, advantages, and features. It is contemplated that various combinations of the stated aspects, advantages and features make up the inventions disclosed in this application.

### BRIEF DESCRIPTION OF DRAWINGS

FIG. 1 is a graph showing the compositional analysis of the product resulting from the liquefaction reaction of HDPE feedstock at 350° C. for 1 hour in the presence of NiMo/H-USY catalyst, NiMo/SiO₂ catalyst, H-USY zeolite, and a catalyst which is a physical mixture of H-USY zeolite and NiMo/SiO₂. It shows the yields of various components (gas (C₁-C₃), naphtha (C₄-C₁₂), kerosene (C₁₃-C₂₀), and C₂₀₊) contained in the product using GC. The solid square for each bar represents the mass balance.

### DETAILED DESCRIPTION OF THE INVENTION

### The Catalyst System

The disclosure provides a novel catalyst system comprising at least one transition metal or transition metal sulfide supported on an oxide-containing support having a low acidity, and an acidic zeolite. The catalyst system of the present disclosure opens new domains for catalyst design as the metals can be supported on different oxide-containing supports to change the chemical and electronic properties of metal sites, without affecting or changing the acid functionality of the zeolites. Another advantage for the catalyst system is that metal catalyst is load on zeolites, it blocks the zeolite pores and decreases the number of available acid sites, but using the system of the present disclosure, the access to acid sites will not be restricted by metal impregnation. The systems also provide improved versatility and easier operability as zeolites may be substituted when poisoned, deactivated, or sintered without compromising the metal load of the catalyst system.

In the context of the present disclosure, the term “low acidity” means that the oxide-containing support has total acid sites of equal or less than about 100 μmol g⁻¹, preferably equal or less than about 90 μmol g⁻¹, preferably equal or less than about 80 μmol g⁻¹, preferably equal or less than about 70 μmol g⁻¹, preferably equal or less than about 60 μmol g⁻¹, 50 μmol g⁻¹, preferably equal or less than about 40 μmol g⁻¹, preferably equal or less than about 30 μmol g⁻¹, preferably equal or less than about 20 μmol g⁻¹, preferably equal or less than about 10 μmol g⁻¹, preferably equal or less than about 5 μmol g⁻¹, preferably equal or less than about 1 μmol g⁻¹, preferably the oxide-containing support has substantially no acidic sites, measured by NH₃-TPD test as provided in the present disclosure. In one or more embodiments, the catalyst support has no acidic sites, measured by NH₃-TPD test.

The metal of the at least one transition metal or transition metal sulfide is typically a Group VI to Group X metal of the periodic table. The metal can be a noble metal (palladium or platinum) or non-noble metal of group VI-A (molybdenum or tungsten) and group VIII-A (cobalt or nickel) of the periodic table. For instance, the metal may be selected from the group consisting of Mo, W, Fe, Co, Ir, Ni, Pd, Pt, or any combination thereof.

In some embodiments, the catalyst system comprises two or more transition metals, transition metal sulfides, or any combination thereof, supported on the oxide-containing support. The metal catalyst may be a blend of two or more different materials within the same type (e.g., two or more different transition metals) or two or more different materials with different types (e.g., one transition metal and one transition metal sulfide). These transition metals or transition metal sulfides are desirable to reactions involving hydrogen, such as hydrocracking, as they provide reaction sites for hydrogen.

In some embodiments, the at least one transition metal or transition metal sulfide is Pt, Pd, Ir, Ni, Fe, Mo, W, Co, NiMo, CoMo, NiW, NiMOSₓ, NiWSₓ or FeSₓ.

In some embodiments, the hydrocracking catalyst is a transition metal sulfide, such as NiMoSₓ, NiWSₓ or FeSₓ.

In some embodiments, the hydrocracking catalyst is bimetallic comprising two different types of transition metal. In one embodiment, the hydrocracking catalyst is bimetallic NiMo. In one embodiment, the hydrocracking catalyst is bimetallic CoMo. In one embodiment, the hydrocracking catalyst is bimetallic NiW.

The catalyst system of the present disclosure has an oxide-containing support with at least one transition metal or transition metal sulfide impregnated over it. The oxide-containing support has a low acidity and may comprise silicon oxides, aluminum oxides, cerium oxides zirconium dioxides, titanium oxides, lanthanum oxides, or any mixture thereof, wherein all the oxidation states of the metals are included in the “oxide” definition. Examples of oxides within this definition include but are not limited to silicon dioxide (SiO₂), aluminum trioxide (Al₂O₃), cerium (IV) dioxide (CeO₂), zirconium dioxide (ZrO₂), titanium dioxide (TiO₂), lanthanum trioxide (La₂O₃), or any mixture thereof. In some embodiments, the oxide-containing support having a low acidity in the catalyst system is silicon oxide.

In some embodiments, the acid zeolite in the catalyst system does not contain metal supported thereon the acid zeolite, for example, the acid zeolite has substantially no active metal sites or even about 0 active metal sites, as measured by a CO titration, as provided in the present disclosure. In the context of the present disclosure, the term “substantially no active metal sites” means that the acid zeolite has total metal sites of equal or less than about 1 μmol g⁻¹, preferably equal or less than about 0.5 μmol g⁻¹, preferably equal or less than about 0.3 μmol g⁻¹, preferably equal or less than about 0.1 μmol g⁻¹, preferably equal or less than about 0.01 μmol g⁻¹, preferably the zeolite has no active metal sites, as measured by CO titration.

A zeolite is a microporous, crystalline aluminosilicate material mainly consisting of silicon, aluminum, and oxygen, typically having the general formula Mⁿ⁺_{1/n} (AlO₂)⁻(SiO₂)ₓ·yH₂O, where Mⁿ⁺_{1/n} is either a metal ion or H⁺, x is Si/Al molar ratio (or SiO2/AlO2 molar ratio) and is greater than 1, and y is the number of water molecules in the formula unit. Any types of zeolites well known to one skilled in the art are suitable herein as support for the transition metal or transition metal sulfide in the hydrocracking catalyst. Exemplary types of zeolites are FAU (e.g., Zeolite X, Zeolite Y, and USY), BEA, MOR, MFI, and FER types. In one or more embodiments, the acid zeolite is a cationic zeolite. In one or more embodiments, the acid zeolite according to the present invention is a protonic zeolite, i.e., M is H+. In one embodiment, H-USY is used as the acid zeolite.

In one or more embodiments the at least one transition metal or transition metal sulfide supported on an oxide-containing support having a low acidity is NiMo supported on silicon oxide (SiO2), and the acid zeolite is H-USY.

In one or more embodiments, the catalyst system is a physical mixture of the at least one transition metal or transition metal sulfide supported on an oxide-containing support and the acid zeolite. In one or more embodiments, the weight ratio of the at least one transition metal or transition metal sulfide supported on an oxide-containing support to the acid zeolite ranges from 1% to 99% wt, of the transition metal or transition metal sulfide supported on an oxide-containing support and 1% to 99% wt, of the acid zeolite. The amount of the transition metal or transition metal sulfide supported on an oxide-containing support may range from a lower limit of 1%, 5%, 10%, 15%, 20%, 25%, 30%, 35%, 40%, 45%, 50%, 55%, 60%, 65%, 70%, 75%, 80% by weight to an upper limit of 50%, 55%, 60%, 65%, 70%, 75%, 80, 85, 90%, 95%, 99% by weight, where any lower limit may be combined with any upper limit, when feasible. The amount of the acid zeolite may range from a lower limit of 1%, 5%, 10%, 15%, 20%, 25%, 30%, 35%, 40%, 45%, 50%, 55%, 60%, 65%, 70%, 75%, 80% by weight to an upper limit of 50%, 55%, 60%, 65%, 70%, 75%, 80, 85, 90%, 95%, 99% by weight, where any lower limit may be combined with any upper limit, when feasible. In one or more embodiments, the weight ratio of the at least one transition metal or transition metal sulfide supported on an oxide-containing support to the acid zeolite ranges from 40% to 99% wt, of the transition metal or transition metal sulfide supported on an oxide-containing support and from 1% to 60% wt, of the acid zeolite, for example from 50% to 90% wt, of the transition metal or transition metal sulfide supported on an oxide-containing support and from 10% wt. to 50% wt, of the acid zeolite. The weight percentages are based on the total weight of the physical mixture. The term “the at least one transition metal or transition metal sulfide supported on an oxide-containing support” refers to the combination of the oxide-containing support and the at least one transition metal or transition metal sulfide.

The catalyst system disclosed herein is useful for converting a carbon-containing feedstock into an alkane-containing product stream. Hence, in another aspect, the present disclosure relates to the use of the catalyst system for converting a carbon-containing feedstock into an alkane-containing product stream. In one or more embodiments, the carbon-containing feedstock is converted into an alkane-containing product stream in a one-pot process.

### Catalyst Synthesis

The catalyst system according to the present disclosure comprises at least one transition metal or transition metal sulfide supported on an oxide-containing support having a low acidity, and an acidic zeolite.

The at least one transition metal or transition metal sulfide supported on an oxide-containing support having a low acidity may be prepared by any method known in the art to synthesize supported metal catalysts. In one or more embodiments, the at least one transition metal or transition metal sulfide supported on an oxide-containing support having a low acidity is prepared by sequential wetness impregnation method.

In one or more embodiments, the preparation of the at least one transition metal or transition metal sulfide supported on an oxide-containing support having a low acidity is made by combining at least one transition metal or transition metal sulfide precursor and an oxide-containing support having a low acidity to form a catalyst precursor mixture. The preparation may also include heating the catalyst precursor mixture.

In one or more embodiments, the at least one transition metal or transition metal sulfide precursor includes at least one transition metal precursor selected from the group consisting of transition metal precursors, rare-earth metal precursors, alkaline-earth metal precursors or a mixture thereof. In one or more embodiments the metal catalyst precursor includes a mixture of transition metal precursor and rare-earth metal precursor. In one or more embodiments the metal catalyst precursor includes a mixture of transition metal precursor, rare-earth metal precursor and alkaline-earth metal precursor.

It is envisioned that the metal catalyst precursor mixture may comprise the at least one transition metal be impregnated into the low acidity support in the form of a metal compound such as transition metal compounds or alloys formed between one or more metals according to the present invention. The formation of transition metal sulfides may be performed by treating the transition metal supported on oxide-containing support with hydrogen sulfide or a mercaptan compound, for example H₂S or DMDS. In one or more embodiments, the at least one transition metal is incorporated in the dispersed phase as the oxidized form (i.e., one or more of its cationic forms), or any transition state.

Preferably, the transition metal precursors may be in the form of oxides, hydroxides or salts such as carbonates, nitrates, acetates, metal salts of an organic acid, ammonium salts or mixtures thereof, with the metal being in the oxidized form or any transition state. In particular embodiments, transition metal catalyst precursors according to the present invention may be selected from transition metal oxides, transition metal hydroxides or transition metal salts such as transition metal carbonates, transition metal nitrates, transition metal acetates, transition metal salts of an organic acid, ammonium salts of transition metals, or mixtures thereof, with the metal being in the oxidized form or any transition state.

Examples of transition metal precursors may include transition metal precursors where the transition metal belongs to Group VI to Group X in the periodic table of elements, including but not limited to elements Mo, W, Fe, Co, Ir, Ni, Pd, Pt, and mixtures thereof, with the metal being in the oxidized state or in any transition state. More preferably, the transition metal is selected from Ni, Mo, or their combinations.

In one or more embodiments, the at least one transition metal precursor comprises at least two transition metals, such as two, three, or four transition metals, in a single or separate precursors.

In one or more embodiments, the at least one transition metal precursor comprises a transition metal carbonate, transition metal acetate, transition metal nitrate or ammonium salt of one or more of Mo, W, Fe, Co, Ir, Ni, Pd, Pt, preferably a transition metal carbonate, transition metal acetate, transition metal nitrate or ammonium salt of one or more of Ni or Mo.

In one or more embodiments, the transition metal precursor may include Ni and Mo. In more particular embodiments, the at least one metal catalyst precursor includes a mixture of nickel nitrate and molybdenium ammonium salt such as ammonium heptamolybdate.

In one or more embodiments, preparing at least one transition metal or transition metal sulfide supported on oxide-containing support having a low acidity combining at least one transition metal precursor and oxide-containing support having a low acidity to form a catalyst precursor mixture, and heating (for example, calcinating) the catalyst precursor mixture to a temperature of about 300° C. to about 750° C. to form the at least one transition metal or transition metal sulfide supported on oxide-containing support having a low acidity. In one or more embodiments, the heating may occur at a temperature of about 350° C. to about 450° C. The formation of transition metal sulfides may be performed by treating the transition metal supported on oxide-containing support with hydrogen sulfide or mercaptan compound, for example H2S or DMDS.

In one or more embodiments, the oxide-containing support having a low acidity may be heat treated before the combination thereof with the at least one transition metal or transition metal sulfide precursor. The heat treatment (for example, calcination) may be made by heating the oxide-containing support to a temperature of about 300° C. to about 750° C., preferably from about 400 to about 600° C.

In one or more embodiments, the at least one transition metal or transition metal sulfide supported on oxide-containing support having a low acidity is mixed with a low acidic zeolite. The mixture may be made by physically mixing according to any method known in the art, for example, by extrusion or compressing the at least one transition metal or transition metal sulfide supported on an oxide-containing support and acid zeolite and make the resulting mixture into pellets.

In one or more embodiments, the weight ratio of the at least one transition metal or transition metal sulfide supported on an oxide-containing support to the acid zeolite ranges from 1% to 99% wt, of the transition metal or transition metal sulfide supported on an oxide-containing support and 1% to 99% wt, of the acid zeolite. The amount of the transition metal or transition metal sulfide supported on an oxide-containing support may range from a lower limit of 1%, 5%, 10%, 15%, 20%, 25%, 30%, 35%, 40%, 45%, 50%, 55%, 60%, 65%, 70%, 75%, 80% by weight to an upper limit of 50%, 55%, 60%, 65%, 70%, 75%, 80, 85, 90%, 95%, 99% by weight, where any lower limit may be combined with any upper limit, when feasible. The amount of the acid zeolite may range from a lower limit of 1%, 5%, 10%, 15%, 20%, 25%, 30%, 35%, 40%, 45%, 50%, 55%, 60%, 65%, 70%, 75%, 80% by weight to an upper limit of 50%, 55%, 60%, 65%, 70%, 75%, 80, 85, 90%, 95%, 99% by weight, where any lower limit may be combined with any upper limit, when feasible. In one or more embodiments, the weight ratio of the at least one transition metal or transition metal sulfide supported on an oxide-containing support to the acid zeolite ranges from 40% to 99% wt, of the transition metal or transition metal sulfide supported on an oxide-containing support and from 1% to 60% wt, of the acid zeolite, for example from 50% to 90% wt, of the transition metal or transition metal sulfide supported on an oxide-containing support and from 10% wt. to 50% wt, of the acid zeolite. The weight percentages are based on the total weight of the physical mixture. The term “the at least one transition metal or transition metal sulfide supported on an oxide-containing support” refers to the combination of the oxide-containing support and the at least one transition metal or transition metal sulfide.

All above descriptions and all embodiments discussed in the above aspects relating to the catalyst system, particularly concerning the oxide-containing support having a low acidity, acidic zeolite, transition metals, and transition metal sulfides, are applicable to these aspects of the invention relating to the synthesis of the catalyst system.

### Processes for Chemically Treating a Carbon-Containing Feedstock and for Depolymerizing a Polymer-Based Feedstock

One aspect of the invention relates to a process for chemically treating a carbon-containing feedstock. The process comprises contacting the carbon-containing feedstock with a hydrogen-containing stream in the presence of a catalyst system, the catalyst system comprising at least one transition metal or transition metal sulfide supported on an oxide-containing support having a low acidity and an acidic zeolite to produce an alkane-containing product stream. In one or more embodiments, the contacting step is a reacting step.

In one or more embodiments, the catalyst system to be used in the process for chemically treating a carbon-containing feedstock is the one described in the present disclosure, in a way that all above descriptions and all embodiments discussed in the above for the catalyst system are applicable to these aspects of the invention relating a process for chemically treating a carbon-containing feedstock.

In another aspect, the present disclosure relates to a process for depolymerizing a polymer-based feedstock, comprising reacting a polymer-based feedstock with a hydrogen-containing stream in the presence of a catalyst system, the catalyst system comprising: at least one transition metal or transition metal sulfide supported on an oxide-containing support having a low acidity, and an acidic zeolite to depolymerize the polymer-based feedstock and form an alkane-containing product stream.

In one or more embodiments, the reaction is a one-step hydrocracking reaction. In one or more embodiments, the one-step hydrocracking reaction does not involve the pyrolysis of the polymer-based feedstock. In one or more embodiments the process is a one-pot process.

In one or more embodiments, the catalyst system is the one described in the present disclosure, in a way that all above descriptions and all embodiments discussed in the above for the catalyst system are applicable to these aspects of the invention relating a process for depolymerizing a polymer-based feedstock.

The metal of the at least one transition metal or transition metal sulfide is typically a Group VI to Group X metal of the periodic table. The metal can be a noble metal (palladium or platinum) or non-noble metal of group VI-A (molybdenum or tungsten) and group VIII-A (cobalt or nickel) of the periodic table. For instance, the metal in the hydrocracking catalyst may be Mo, W, Fc, Co, Ir, Ni, Pd, Pt, or any combination thereof.

In some embodiments, the hydrocracking catalyst comprises two or more transition metals, transition metal sulfides, or any combination thereof, supported on the oxide-containing support. The catalyst may be a blend of two or more different materials within the same type (e.g., two or more different transition metals) or two or more different materials with different types (e.g., one transition metal and one transition metal sulfide). These transition metals or transition metal sulfides are desirable to reactions involving hydrogen, such as hydrocracking, as they provide reaction sites for hydrogen.

In some embodiments, the at least one transition metal or transition metal sulfide is Pt, Pd, Ir, Ni, Fe, Mo, W, Co, NiMo, CoMo, NiW, NiMOSₓ, NiWSₓ or FeSₓ. In some embodiments, the hydrocracking catalyst is a transition metal sulfide, such as NiMoSₓ, NiWSₓ or FeSₓ. In some embodiments, the hydrocracking catalyst is bimetallic comprising two different types of transition metal. In one embodiment, the hydrocracking catalyst is bimetallic NiMo. In one embodiment, the hydrocracking catalyst is bimetallic CoMo. In one embodiment, the hydrocracking catalyst is bimetallic NiW.

The catalyst system of the present disclosure has an oxide-containing support with at least one transition metal or transition metal sulfide impregnated over it. The oxide-containing support has a low acidity and may comprise silicon oxides, aluminum oxides, cerium oxides zirconium dioxides, titanium oxides, lanthanum oxides, or any mixture thereof, wherein all the oxidation states of the metals are included in the “oxide” definition. Examples of oxides within this definition include but are not limited to silicon dioxide (SiO₂), aluminum trioxide (Al₂O₃), cerium (IV) dioxide (CeO₂), zirconium dioxide (ZrO₂), titanium dioxide (TiO₂), lanthanum trioxide (La₂O₃), or any mixture thereof.

In some embodiments, the acid zeolite in the catalyst system does not contain metal supported thereon the acid zeolite, for example, the acid zeolite has substantially no active metal sites or even about 0 active metal sites, as measured by a CO titration as per the method provided in the present disclosure. In the context of the present disclosure, the term “substantially no active metal sites” means that the acid zeolite has total acid sites of equal or less than about 1 μmol g⁻¹, preferably equal or less than about 0.5 μmol g⁻¹, preferably equal or less than about 0.3 μmol g⁻¹, preferably equal or less than about 0.1 μmol g⁻¹, preferably equal or less than about 0.01 μmol g⁻¹, preferably the zeolite has no active metal sites, as measured by CO titration.

A zeolite is a microporous, crystalline aluminosilicate material mainly consisting of silicon, aluminum, and oxygen, typically having the general formula Mⁿ⁺_{1/n} (AlO₂)⁻(SiO₂)ₓ·yH₂O, where Mⁿ⁺_{1/n} is either a metal ion or H⁺, x is Si/Al molar ratio (or SiO2/AlO2 molar ratio) and is greater than 1, and y is the number of water molecules in the formula unit. Any types of zeolites well known to one skilled in the art are suitable herein as support for the transition metal or transition metal sulfide in the hydrocracking catalyst. Exemplary types of zeolites are FAU (e.g., Zeolite X, Zeolite Y, and USY), BEA, MOR, MFI, and FER types. In one or more embodiments, the acid zeolite is a cationic zeolite. In one or more embodiments, the acid zeolite according to the present invention is a protonic zeolite, i.e., M is H+. In one embodiment, H-USY is used as the acid zeolite.

In one or more embodiments the at least one transition metal or transition metal sulfide supported on an oxide-containing support having a low acidity is NiMo supported on silicon oxide (SiO₂), and the acid zeolite is H-USY.

In one or more embodiments, the catalyst system is a physical mixture of the at least one transition metal or transition metal sulfide supported on an oxide-containing support and acid zeolite. The mixture physical mixture may be made according to any method known in the art, for example, by extrusion or compressing the at least one transition metal or transition metal sulfide supported on an oxide-containing support and acid zeolite and make the resulting mixture into pellets. In one or more embodiments, the weight ratio of the at least one transition metal or transition metal sulfide supported on an oxide-containing support to component acid zeolite ranges from about 2:1 to about 1:2, such as from about 1.5:1 to about 1:1.5, from about 1.2:1 to about 1:1.2, or from about 1.2:1 to about 1:1.

The feedstock used in the processes for chemically treating a carbon-containing feedstock described herein can be any carbon-containing feedstock, for example, a polymer-based feedstock.

The carbon-containing feedstock (e.g., a polymer-based feedstock) may be a petroleum-based resin (e.g., petroleum-based virgin resin), bio-based resin, recycled resin, or combinations thereof. For instance, the carbon-containing feedstock (e.g., a polymer-based feedstock) may comprise a virgin resin, a recycled resin, or combinations thereof. In some embodiments, the carbon-containing feedstock (e.g., a polymer-based feedstock) may comprise a combination of a recycled resin, biobased resin, and optionally a petroleum-based resin such that the resulting composition achieves low or neutral carbon emission (or even a carbon uptake).

The recycled resin may comprise a post-consumer resin (PCR), a post-industrial resin (PIR), or combinations thereof, including regrind, scraps and defective articles. PCR refers to resins that are recycled after consumer use, whereas PIR refers to resins that are recycled from industrial materials and/or processes (for example, cuttings of materials used in making other articles). The recycled resin may include resins having been used in rigid applications (such as from blow molded articles, including 3D-shaped articles) as well as in flexible applications (such as from films). The recycled resin may be of any color, including, but not limited to, black, white, or grey, depending on the color used in the ultimate article. The form of the recycled resin is not particularly limited, and may be in pellets, flakes, and agglomerated films. In some embodiments, the recycled resin used is a PCR or PIR that comprises one or more polyolefins. In some embodiments, the recycled resin is a recycled material according to ISO 14021. In some embodiments, the carbon-containing feedstock (e.g., a polymer-based feedstock) is a post-consumer resin (PCR) or a post-industrial resin (PIR).

In some embodiments, the carbon-containing feedstock is a polymer-based feedstock. Exemplary polymer-based feedstocks are polyolefins, polyvinyl chlorides, polyesters, polystyrenes, polyacrylates, polymethacrylates, polyamides, polycarbonates, and mixtures thereof.

Suitable polyolefins include those prepared from linear, branched, or cyclic olefin monomers having 2 to 20 carbon atoms, 2 to 16 carbon atoms, or 2 to 12 carbon atoms. Exemplary olefin monomers are α-olefins including but not limited to ethylene, propylene, 1-butene, 2-butene, 1-pentene, 3-methyl-1-butene, 1-hexene, 4-methyl-1-pentene, 3-methyl-1-pentene, 4,6-dimethyl-1-heptene, 1-octene, 1-nonene, 1-decene, 1-undecene, 1-dodecene, 1-tetradecene, 1-hexadecene, 1-octadecene, 1-eicocene, vinylcyclohexane, styrene, tetracyclododecene, norbornene, 5-ethylidene-2-norbornene (ENB), and combinations thereof. These olefins may each contain a heteroatom such as an oxygen, nitrogen, or silicon atom.

Exemplary polyolefins include a propylene-based polymer, an ethylene-based polymers, an ethylene-vinyl ester polymer, or a C₄-C₁₂ olefin-based polymer. The ethylene-based polymer contained can be low-density polyethylene (LDPE), linear low-density polyethylene (LLDPE), high-density polyethylene (HDPE), medium-density polyethylene (MDPE), polyethylene wax, ultrahigh-molecular weight polyethylene, ethylene copolymer, and combinations thereof.

Suitable polyolefins also include a copolymer prepared from two or more olefin comonomers, which include polyene comonomers (having 3 to 20 carbon atoms including but not limited to butadiene (e.g., 1,3-butadiene), isoprene, pentadiene (e.g., 1,3-pentadiene; 1,4-pentadiene; 3-methyl-1,4-pentadiene; 3,3-dimethyl-1,4-pentadiene), dimethylbutadiene, dimethylpentadiene, hexadiene (e.g., 1,3-hexadiene; 1,4-hexadiene; 1,5-hexadiene; 4-methyl-1,4-hexadiene; 5-methyl-1,4-hexadiene; 3-methyl-1,5-hexadiene; 3,4-dimethyl-1,5-hexadiene), heptadiene (e.g., 1,3-heptadiene; 1,4-heptadiene; 1,5-heptadiene; 1,6-heptadiene; 6-methyl-1,5-heptadiene), methylhexadiene, dimethylhexadiene, octadiene (e.g., 1,3-octadiene; 1,4-octadiene; 1,5-octadiene; 1,6-octadiene; 1,7-octadiene; 7-methyl-1,6-octadiene; 3,7-dimethyl-1,6-octadiene; 5,7-dimethyl-1,6-octadiene), nonadienes (e.g., 1,8-nonadiene), decadiene (e.g., 1,9-decadiene), undecadiene (e.g., 1,10-undecadiene), dicyclopentadienes, octatriene (e.g., 3,7,11-trimethyl-1,6,10 octatriene), 4-vinyl cyclohexene, dicyclopentadiene, vinyl comonomers (including but not limited to acrylonitrile and acrylamide, and their derivatives), and vinylaromatic comonomers (including but not limited to styrene and its derivatives, such as α-methylstyrene); any of which may each contain a heteroatom such as an oxygen, nitrogen, or silicon atom.

Suitable styrene-based polymers include but are not limited to polymers prepared from monomers such as styrene, α-methylstyrene, p-methylstyrene, vinylxylene, vinylnaphthalene, and mixtures thereof; and optionally a diene comonomer such as butadiene, isoprene, pentadiene, and mixtures thereof.

Suitable polyesters include but are not limited to polyethylene terephthalate, polybutylene terephthalate, polyethylene naphthalate, polybutylene naphthalate, polyethylene isophthalate, polycarbonate, copolymerization of polyesters with ethylene terephthalate as a main repeating unit (such as polyethylene (terephthalate/isophthalate), polyethylene (terephthalate/isophthalate), polyethylene (terephthalate/adipate), polyethylene (terephthalate/sodium sulfoisophthalate), polyethylene (terephthalate/sodium isophthalate), polyethylene (terephthalate/phenyl-dicarboxylate) and polyethylene (terephthalate/decane dicarboxylate)), and copolymerization of polyesters with a butylene terephthalate as a main repeating unit (such as polybutylene (terephthalate/isophthalate)), polybutylene (terephthalate/adipate), polybutylene (terephthalate/sebacate), polybutylene (terephthalate/decane dicarboxylate)).

Suitable polyamides include aliphatic polyamides such as nylon-6, nylon-66, nylon-10, nylon-12 and nylon-46; and aromatic polyamides produced from aromatic dicarboxylic acid and aliphatic diamine.

The carbon-containing feedstock (e.g., a polymer-based feedstock) may be plastic materials containing, but are not limited to, ABS, polyacetal, acrylic, ionomer, polyamide in general, Nylon 6, Nylon 6/6, Nylon 6/9, Nylon 6/10, Nylon 6/12, Nylon 11, Nylon 12, polycarbonate, polyester (PBT), polyester (PET), polyether ether ketone, polyethylene, polyolefin in general, polyphenylene ether, polyphenylene sulfide, polypropylene, polystyrene, polysulfone, polyurethane, SAN and thermoplastic elastomer. In some embodiments, the carbon-containing feedstock contain intermediate thermoplastics, such as polymethyl methacrylate, acrylonitrile-butadiene-styrene, acrylonitrile/acrylate/styrene, acrylonitrile/ethylene-propylene-diene monomer (EPDM)/styrene, styrene/maleic anhydride copolymers and rubber blends, cellulose-acetate-butyral, thermoplastic olefin elastomer, and the like.

The reaction is typically performed at moderate temperatures, ranging from about 200° C. to 500° C. For instance, the reaction temperature may range from about 280° C. to 470° C., from about 280° C. to about 450° C., from about 280° C. to about 430° C., from about 280° C. to about 410° C., from about 280° C. to about 400° C., from about 280° C. to about 370° C., from about 280° C. to about 340° C., from about 280° C. to about 310° C., from about 300° C. to 470° C., from about 300° C. to about 450° C., from about 300° C. to about 430° C., from about 300° C. to about 410° C., from about 300° C. to about 400° C., from about 300° C. to about 370° C., from about 300° C. to about 340° C., from about 310° C. to 470° C., from about 310° C. to about 450° C., from about 310° C. to about 430° C., from about 310° C. to about 410° C., from about 310° C. to about 400° C., from about 310° C. to about 370° C., from about 310° C. to about 360° C. In one embodiment, the reaction temperature ranges from about 310° C. to about 360° C.

The reaction is typically performed at a hydrogen pressure ranging from about 1 bar to about 200 bar. For instance, the reaction hydrogen pressure may range from about 5 bar to about 100 bar, from about 5 bar to about 90 bar, from about 5 bar to about 80 bar, from about 5 bar to about 70 bar, from about 5 bar to about 60 bar, from about 10 bar to about 100 bar, from about 10 bar to about 90 bar, from about 10 bar to about 80 bar, from about 10 bar to about 70 bar, from about 10 bar to about 60 bar, from about 20 bar to about 100 bar, from about 20 bar to 90 bar, from about 20 bar to 80 bar, from about 20 bar to about 70 bar, from about 20 bar to about 60 bar, from about 30 bar to about 100 bar, from about 30 bar to about 90 bar, from about 30 bar to about 80 bar, from about 30 bar to about 70 bar, from about 30 bar to about 60 bar, about 40 bar to about 100 bar, from about 40 bar to about 90 bar, from 40 bar to 80 bar, from 40 bar to 70 bar, from 40 bar to 60 bar, or from about 45 bar to about 55 bar.

The products obtained from the process described herein include saturated hydrocarbons, such as alkanes, and optionally unsaturated hydrocarbons, such as unsaturated acyclic hydrocarbons (e.g., alkenes) and/or aromatics.

The product streams obtained from the hydrocracking reaction include a liquid product stream (primarily C₄-C₂₀ hydrocarbons, e.g., naphtha or naphtha-like product, and/or diesel, kerosene or kerosene-like product), an optional gas product stream (primarily C₁-C₃ hydrocarbons), and an optional solid product stream (primarily C₂₀₊ hydrocarbons).

In some embodiments, the process produces an alkane-containing product stream comprises C₁-C₂₀ hydrocarbons. In one or more embodiments, the C₁-C₂₀ hydrocarbons are C₁-C₂₀ alkanes.

The process described herein efficiently cracks carbon-containing feedstock, while increasing the yield and quality of the recovered liquid hydrocarbon product suitable for further cracking. The process can have a C₁-C₂₀ hydrocarbon, for example, C₁-C₂₀ alkane, product yield of at least 50%. For instance, the process can have a C₁-C₂₀ hydrocarbon (for example a C₁-C₂₀ alkane) product yield of at least 55%, at least 60%, at least 65%, at least 70%, at least 75%, at least 80%, at least 85%, at least 90%, or at least 95% by weight, based on the total weight of the resulting product.

The process of the present disclosure can also selectively produce an alkane-containing product stream containing an increased amount of naphtha or naphtha-like product compared to a process that does not employ the catalyst system as described herein (having otherwise same conditions). “Naphtha” or “naphtha-like” product may include hydrocarbons or mixtures thereof, majority of which having a carbon chain length ranging from C₄ to C₁₄, C₅ to C₁₄, C₄-C₁₂, or C₅-C₁₂. In one embodiment, the naphtha or naphtha-like product contains primarily C₄-C₁₂, hydrocarbons, for example contains C₄-C₁₂ alkanes.

In some embodiments, the process has a selectivity towards naphtha or naphtha-like product of at least 50%, for instance, at least 55%, at least 60%, at least 65%, at least 70%, at least 75%, at least 80%, at least 85%, at least 90%, at least 95%, at least 96%, at least 97%, at least 99%, at least 99%, or virtually 100%, by weight, based on the total weight of the product stream.

In some embodiments, the process produces less than 10%, such as less than 5%, less than 3% or less than 1% by weight of solid product stream such as C₂₀₊ hydrocarbons, and/or wherein the process produces less than 10%, such as less than 5%, less than 3% or less than 1%, by weight of diesel, kerosene or kerosene-like product, such as C₁₃-C₂₀ hydrocarbons.

After converting the carbon-containing feedstock (e.g., a polymer-based feedstock) to an alkane-containing product stream, the product stream may be further separated into two or more different product streams. The separation may be based on the molecular weight of the components of the alkane-containing product stream. Thus, the process may further comprise the step of separating the alkane-containing product stream into two or more different product streams based on the molecular weight of the components of the alkane-containing product stream.

In some embodiments, the process may further comprise the step of separating the product stream into two or more different product streams such as a light-component stream (e.g., a gas stream, such as C₁-C₃ product stream or a C₁-C₄ product stream), a naphtha or naphtha-like stream (e.g., a C₄-C₁₂ product stream, C₅-C₁₂ product stream, C₄-C₁₄ product stream, or C₅-C₁₄ product stream), a diesel or kerosene product stream (e.g., a C₁₃-C₁₉ product stream, C₁₃-C₂₀ product stream, C₁₅-C₁₉ product stream, or C₁₅-C₂₀ product stream), and/or a heavy-component stream (e.g., a C₂₀₊ product stream such as C₂₀ to Cso product stream). In one embodiment, the diesel or kerosene product stream contains primarily C₁₃-C₂₀, hydrocarbons.

In one embodiment, the different product streams are selected from a C₁-C₃ product stream, a C₄-C₁₂ product stream, a C₁₃-C₂₀ product stream, and a C₂₀₊ product stream.

In some embodiments, the process may further comprise the step of pre-mixing the carbon-containing feedstock (e.g., the polymer-based feedstock) with a solvent medium, prior to the contacting or reacting step. The solvent medium typically is a liquid that can act as a medium to transport the polymer-based feedstock (e.g., a hot, viscous plastic waste) to the reactor.

In some embodiments, the solvent medium comprises a liquid hydrocarbon.

The solvent medium may come from the liquid product stream that is produced and/or separated from the process described herein. For instance, the liquid product stream (e.g., C₄-C₂₀ hydrocarbons), formed from the process described herein and separated from the gas product stream and/or solid product stream as described herein, can be used as the solvent medium to mix with the polymer-based feedstock and transport the polymer-based feedstock to the reactor. As another example, diesel, kerosene or kerosene like product stream (e.g., C₁₃-C₂₀ hydrocarbons), formed from the process described herein and separated from the gas product stream, naphtha or naphtha like product stream, and/or solid product stream as described herein, can be used as the solvent medium to mix with the polymer-based feedstock and transport the polymer-based feedstock to the reactor. Thus, in some embodiments, the solvent medium comprises a liquid product stream (e.g., C₄-C₂₀ hydrocarbons), obtained from the depolymerization reaction. In some embodiments, the solvent medium comprises a diesel, kerosene or kerosene-like product stream (e.g., C₁₃-C₂₀ hydrocarbons), obtained from the depolymerization reaction. The solvent medium may come from the liquid product stream.

Additional aspects of the invention relate to various products or product streams produced from the process described herein. Thus, one aspect of the invention relates to an alkane-containing mixture obtained from the processes described herein. In some embodiments, the disclosure provides an alkane-containing mixture obtained from the processes for chemically treating a carbon-containing feedstock described herein. In some embodiments, the disclosure provides an alkane-containing mixture obtained from the process for depolymerizing a polymer-based feedstock described herein.

All above descriptions and all embodiments discussed in the above aspects relating to the process for chemically treating a carbon-containing feedstock and the process for depolymerizing a polymer-based feedstock, including various aspects of the feedstock, the hydrogen stream, the hydrocracking catalyst, and the hydrocracking reaction conditions are applicable to this aspect of the invention relating to an alkane-containing mixture obtained from the above processes.

In some embodiments, the alkane-containing mixture obtained from the process described herein contains at least 50%, at least 55%, at least 60%, at least 65%, at least 70%, at least 75%, at least 80%, at least 85%, at least 90%, or at least 95%, by weight, of C₁-C₂₀ hydrocarbons (e.g., C₁-C₂₀ alkanes), based on the total weight of the alkane-containing mixture obtained from the process.

In some embodiments, the alkane-containing mixture obtained from the process described herein contains at least 50%, for instance, at least 55%, at least 60%, at least 65%, at least 70%, at least 75%, at least 80%, at least 85%, at least 90%, at least 95%, at least 96%, at least 97%, at least 99%, at least 99%, or virtually 100%, by weight, of naphtha or naphtha-like product (e.g., C₄-C₁₄, C₅ to C₁₄, C₄-C₁₂, or C₅-C₁₂ hydrocarbons; such as C₄ to C₁₄, C₅ to C₁₄, C₄-C₁₂, or C₅-C₁₂ alkanes) based on the total weight of the alkane-containing mixture obtained from the process. In one embodiment, the naphtha or naphtha-like product contains primarily C₄-C₁₂, hydrocarbons, e.g., C₄-C₁₂ alkanes.

In some embodiments, the mixture comprises less than 10%, such as less than 5%, less than 3% or less than 1% by weight of solid product stream such as C₂₀₊ hydrocarbons, and/or wherein the process produces less than 10%, such as less than 5%, less than 3% or less than 1%, by weight of diesel, kerosene or kerosene-like product (such as C₁₃-C₂₀ hydrocarbons).

The alkane-containing mixture obtained from the process described herein can be further cracked to monomers, separated into various monomer components, and/or further fed to a polymerization unit for preparing polymers.

Thus, the process may further comprise a step of converting the alkane-containing mixture (e.g., the naphtha or naphtha-like product containing primarily C₄-C₁₂, hydrocarbons) obtained from the process described herein to produce olefins and/or aromatics. In some embodiments, the optional converting step comprises thermally cracking the alkane-containing mixture (e.g., the naphtha or naphtha-like product containing primarily C₄-C₁₂, hydrocarbons) obtained from the process described herein to obtain one or more lower hydrocarbons. In one embodiment, the alkane-containing mixture (e.g., the naphtha or naphtha-like product containing primarily C₄-C₁₂, hydrocarbons) obtained from the process described herein is mixed with high-pressure steam and fed through a furnace for the cracking step.

In some embodiments, the optional converting step results in a mixture of lower hydrocarbons comprising one or more of ethylene, propylene, butadiene, 1-butene, C₅ crude (i.e., a mixture of C₅ components), C-5 dienes crude (i.e., a mixture of C₅ components rich in cyclopentadiene, piperylene and isoprene), isoprene, and aromatics (e.g., benzene, toluene, xylenes, and cumene). In one embodiment, the optional converting step results in ethylene. In one embodiment, the optional converting step results in propylene. In one embodiment, the optional converting step results in ethylene and propylene.

In some embodiments, the process may further comprise a step for separating the monomer components produced from the converting step described above. In one embodiment, the process may further comprise a step for separating ethylene from other components produced from the converting step described above. In one embodiment, the process may further comprise a step for separating propylene from other components produced from the converting step described above. In one embodiment, the process may further comprise a step for separating ethylene and propylene from other components produced from the converting step described above.

In some embodiments, the process may further comprise a step of polymerizing the monomer components produced from the converting step and/or separating step described above, to form a polyolefin. In one embodiment, the process may further comprise a step of polymerizing ethylene produced from the converting step and/or separating step described above, to form a polyolefin. In one embodiment, the process may further comprise a step of polymerizing propylene produced from the converting step and/or separating step described above, to form a polyolefin. In one embodiment, the process may further comprise a step of polymerizing ethylene and/or propylene produced from the converting step and/or separating step described above, to form a polyolefin.

### The System for Carrying Out the Process

Another aspect of the invention relates to a system/apparatus for chemically treating a carbon-containing feedstock. The system/apparatus comprises a reactor receiving the carbon-containing feedstock (e.g., a polymer-based feedstock), a hydrogen stream, and the catalyst system of the present disclosure, wherein the reactor is configured to convert the carbon-containing feedstock (e.g., a polymer-based feedstock) into an alkane-containing product stream.

Another aspect of the invention relates to a system/apparatus for depolymerizing a polymer-based feedstock. The system/apparatus comprises a reactor receiving the polymer-based feedstock, a hydrogen stream, and the catalyst system of the present diclosure, wherein, in the reactor, the polymer-based feedstock reacts with the hydrogen stream in the presence of the catalyst system. Such reaction may be carried out in a one-step hydrocracking reaction, to depolymerize the polymer-based feedstock and form an alkane-containing product stream.

All above descriptions and all embodiments discussed in the above aspects relating to the process for chemically treating a carbon-containing feedstock and the process for depolymerizing a polymer-based feedstock, including various aspects of the feedstock, the hydrogen stream, the hydrocracking catalyst, and the hydrocracking reaction conditions are applicable to these aspects of the invention relating to a system/apparatus for chemically treating a carbon-containing feedstock or a system/apparatus for depolymerizing a polymer-based feedstock.

The reaction can be carried out in a heterogeneous reactor, which can comprise one or more reactor inlets for receiving the carbon-containing feedstock (e.g., polymer-based feedstock), a hydrogen stream, and at least one hydrocracking catalyst; and one or more reactor outlets for outputting the product stream. The heterogeneous reactor can contain a catalytic bed for holding the catalyst system. The reactor can further contain a heating module for controlling the reaction temperature. The reactor can further contain a pressure control module for controlling the hydrogen pressure during the reaction.

The reactor may be an ebullated reactor, a slug flow reactor, or a fixed bed reactor. The choice of the reactor for operating the process may be determined by the choice of the catalyst system. For instance, a slug flow reactor may be used with a hydrocracking catalyst containing iron; and an ebullated bed reactor or a fixed bed reactor may be used with a catalyst containing Ni, Co, or Mo, e.g., NiMo, or CoMo catalysts.

In some embodiments, two or more different reactors can be used in series, to further hydrogenate/treat the products. For instance, an ebullated bed reactor may be used first for hydrocracking reaction, followed by a fixed bed reactor that can further remove contaminants, such as sulfur, nitrogen, etc.

In some embodiments, the system/apparatus has a single reactor.

The reactor may also be a continuous reaction, e.g., any form of a continuous flow reactor.

As discussed above, the reaction herein may employ a single-stage, hydrocracking unit, i.e., the cracking and hydrotreating of the carbon-containing feedstock (e.g., a polymer-based feedstock) are carried out in one-step. In one or more embodiments, the reaction avoids a pyrolysis reaction. In some embodiments, the system/apparatus does not include a pyrolysis unit.

The system/apparatus may further comprise a separator downstream the reactor to separate the product stream (e.g., the alkane-containing product stream) into two or more different product streams. The separator may operate to separate different product streams from each other based on the molecular weight of the components of the product stream (e.g., the alkane-containing product stream).

The separator may be a gas separator, a cyclone, a flash vessel, or a distillation column.

The separated light-component stream (such as C₁-C₃ product stream or a C₁-C₄ product stream) can be used as a source of hydrogen stream, by using a steam reformer and shift reactor. The resulting hydrogen stream can be re-directed to the reactor for the hydrocracking reaction.

### Other Methods and Uses

Certain aspects of the invention relate to methods for controlling a carbon chain length distribution of a product stream obtained from chemically treating a carbon-containing feedstock or depolymerizing a polymer-based feedstock, such as selectively converting the carbon-containing feedstock or polymer-based feedstock to a liquid naphtha or naphtha-like product.

In some embodiments, the disclosure provides a method for controlling an alkane carbon chain distribution of a product stream obtained from depolymerizing a polymer-based feedstock. The method comprises the steps of reacting a polymer-based feedstock with a hydrogen-containing stream in the presence of a catalyst system comprising at least one transition metal or transition metal sulfide supported on an oxide-containing support having a low acidity and an acidic zeolite, to chemically treat a carbon-containing feedstock or to depolymerize the polymer-based feedstock and form a liquid naphtha or naphtha-like product containing at least 50% (such as at least 65%, at least 80%, at least 85%, at least 90%, or at least 95%) by weight of C4-C12 hydrocarbons (e.g., C4-C12 alkanes); less than 10% (such as less than 5%, less than 3% or less than 1%) by weight of solid product stream (such as C20+ hydrocarbons); and optionally less than 10% (such as less than 5%, less than 3% or less than 1%) by weight of diesel, kerosene or kerosene like product (such as C13-C20 hydrocarbons), wherein the reaction temperature ranges from about 300-450° C. and hydrogen pressure ranges from about 5 to 100 bar.

All above descriptions and all embodiments discussed in the above aspects relating to the process for chemically treating a carbon-containing feedstock and the process for depolymerizing a polymer-based feedstock, including various aspects of the feedstock, the hydrogen stream, the hydrocracking catalyst, and the hydrocracking reaction conditions are applicable to these aspects of the invention relating to a method for selectively converting a polymer-based feedstock to a liquid naphtha or naphtha-like product.

Another aspect of the invention relates to a method comprising utilizing the catalyst system described herein for converting a carbon-containing feedstock into an alkane-containing product stream.

Another aspect of the invention relates to a method comprising utilizing the catalyst system described herein for depolymerizing a polymer-based feedstock into an alkane-containing product stream.

In some embodiments, the catalyst system described herein is utilized in a one-step process for converting a carbon-containing feedstock into an alkane-containing product stream or a one-step process for depolymerizing a polymer-based feedstock into an alkane-containing product stream.

In some embodiments, the catalyst system described herein is utilized in a one-pot process for converting a carbon-containing feedstock into an alkane-containing product stream or a one-pot process for depolymerizing a polymer-based feedstock into an alkane-containing product stream.

All above descriptions and all embodiments discussed in the above aspects relating to the process for chemically treating a carbon-containing feedstock and the process for depolymerizing a polymer-based feedstock, including various aspects of the feedstock, the hydrogen stream, the hydrocracking catalyst, and the hydrocracking reaction conditions are applicable to these aspects of the invention relating to methods for utilizing the catalyst system for converting a carbon-containing feedstock into an alkane-containing product stream or for depolymerizing a polymer-based feedstock into an alkane-containing product stream.

Another aspect of the invention relates to a method comprising utilizing the alkane-containing mixture (e.g., the liquid naphtha or naphtha-like product stream) obtained from the processes described herein for a secondary use. That secondary use may be storage (literally) or include any other viable secondary use, such as further processing or immediate use, e.g., for further application such as being directly fed into a chemical cracking furnace to produce value-adding chemicals, such as productions of olefin monomers (e.g., ethylene and/or propylene), and/or further polymerization thereof.

All above descriptions and all embodiments discussed in the above aspects relating to the process for chemically treating a carbon-containing feedstock and the process for depolymerizing a polymer-based feedstock, including various aspects of the feedstock, the hydrogen stream, the hydrocracking catalyst, and the hydrocracking reaction conditions are applicable to these aspects of the invention relating to methods for utilizing a hydrocracking catalyst for converting a carbon-containing feedstock into an alkane-containing product stream or for depolymerizing a polymer-based feedstock into an alkane-containing product stream.

All above descriptions and all embodiments discussed in the above aspects relating to various products or product streams produced from the process described herein, such as the alkane-containing mixture obtained from the processes described herein, are applicable to this aspect of the invention relating to methods of utilizing the alkane-containing mixture obtained from the processes described herein for a secondary use.

### EXAMPLES

The following examples are for illustrative purposes only and are not intended to limit, in any way, the scope of the present invention.

### Example A—Synthesis of an Exemplary Catalyst System (NiMo/H-USY, NiMo/γ-Al₂O₃ and NiMo/SiO2)

NiMo-supported catalysts were synthesized using the sequential wetness impregnation method. Before synthesis, the H-USY zeolite (Zeolyst International), and the SiO₂ catalyst supports (17.5 g of each) were calcined at 550° C. for 4 hours under air. Prior to synthesis, 2.5 g of nickel nitrate and 3.7 g of ammonium heptamolybdate metal precursors were dissolved separately in deionized water to make an aqueous solution, based on the desired Ni to Mo weight ratio of 2.5% wt and 10.0% wt, respectively. The molybdenum precursor solution was impregnated into the calcined support, followed by overnight drying at 110° C. After drying, the nickel precursor solution was impregnated, followed by overnight drying at 110° C. and calcination at 550° C. for 4 hours under air.

### Example B—Catalyst Characterization-Acid and Metal Sites

AuctoChem II instrument was used for the sample NH₃ TPD titration (acid sites) and CO titration (metal sites). For both characterizations about 100 mg of NiMo-supported sample was loaded into the reactor, followed by pretreatment at 550° C. for 1 h under the flow of hydrogen. For NH₃ TPD, after pretreatment, the sample was exposed to the flow of 5% NH₃ in Ar, for 1 h at 100° C. After NH₃ exposure, the catalyst was purged under the flow of Ar for 1 h to remove weakly adsorbed NH₃. This step was followed by heating under the flow of Ar to 650° C., at 10° C. min-1. The NH₃ TPD profile was measured using a mass spectrometer, to quantify the sample acid site concentration. For the NH₃ TPD of H-USY zeolite, the 100 mg sample pretreatment was done under the flow of 10% O2 in Ar, for 1 h at 550° C. For the CO titration of the NiMo-supported samples, after H2 pretreatment the sample was exposed to 20 pulses of 10% CO in Ar to titrate the active metal sites. The CO pulses were measured using a TCD detector, to quantify the active metal site concentration. The table below shows the sample acid and active metal site concentrations. H-USY was considered as absent of active metal sites. The results are shown in Table 1.

TABLE 1
Catalyst characterization for NiMo supported
catalysts and H—USY zeolite.

| Catalyst | Metal sites/μmol g⁻¹ | Acid sites/μmol g⁻¹ |
|---|---|---|
| NiMo/H—USY | 13.3 | 515 |
| NiMo/SiO₂ | 9.7 | 0 |
| H—USY | 0 | 453 |

### Example C—Activity Test

In order to test the NiMo/H-USY catalyst, about 400 mg of HDPE and 75 mg of NiMo/H-USY catalyst were loaded inside a microbomb batch reactor. The reactor was purged with He several times before pressurizing with H₂ to 50 bar. The reactor was immersed in a fluidized sand bath held at 350° C. for 1 hour.

In order to test the NiMo/SiO₂ catalyst, about 400 mg of HDPE and 100 mg of NiMo/SiO₂ catalyst were loaded inside a microbomb batch reactor, rest of the reaction conditions were same as above. The amount of NiMo/SiO₂ used for this reaction had a similar amount of active metal sites as in NiMo/H-USY.

In order to test the non-impregnated zeolite (H-USY), about 400 mg of HDPE and 90 mg of H-USY support were loaded inside a microbomb batch reactor, rest of the reaction conditions were the same as the other catalysts. The amount of H-USY used for this reaction had a similar amount of acid sites as in NiMo/H-USY.

In order to test a physical mixture of H-USY zeolite and NiMo/SiO₂, about 400 mg of HDPE was mixed with a physical mixture of 100 mg of NiMo/SiO2 and 90 mg of H-USY, rest of the reaction conditions were the same as above. The amount of NiMo/SiO2 and H-USY used for this reaction had a similar amount of active metal and acid sites as in NiMo/H-USY.

After the reaction, the product yield and composition were analyzed with an Agilent 8890 GC instrument, using Supelco Petrocol DH (100 m×250 μm×0.5 μm) and DB-1 column (30 m×320 μm×1 μm). The product quality was analyzed with a Gerstel GC-MS instrument, using PoraBOND (100 m×250 μm×0.5 μm), DB-1 (30 m×320 μm×1 μm) and GS-GasPro (30 m×320 μm×0 μm) column.

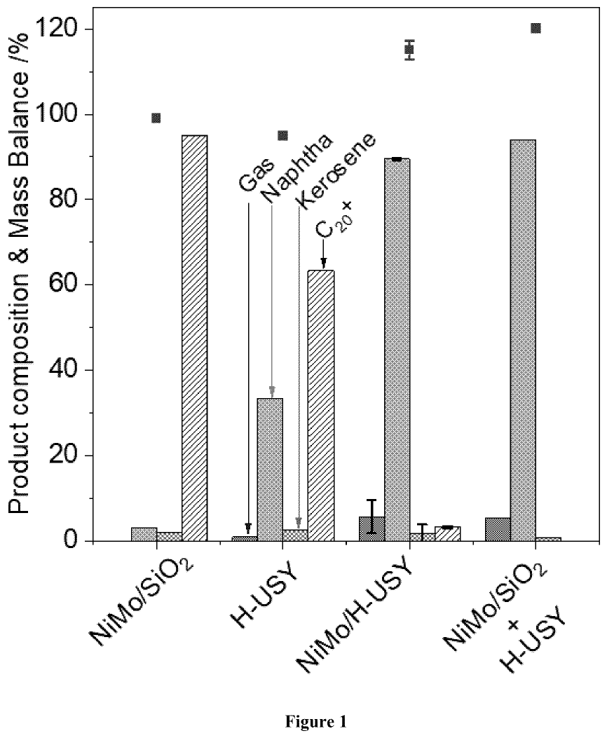

The results are shown in FIG. 1. The chart shows that the NiMo/SiO2 catalyst gave lower naphtha yield and more than 95% yield for C20+ or unreacted HDPE. With the H-USY support the naphtha yield was higher than that with NiMo/SiO2, but there was a significant amount of C20+ products. With NiMo/H-USY the naphtha yield was more than 90%, suggesting that both the metal and acid functionalities are required to increase the naphtha yield. The physical mixture of NiMo/SiO2+H-USY gave a similar naphtha yield as obtained with NiMo/H-USY, showing that the close proximity of metal and acid functionalities is not a necessity for HDPE liquefaction to naphtha.

1. A catalyst system, comprising
(a) at least one transition metal or transition metal sulfide supported on an oxide-containing support having a low acidity, and
(b) an acidic zeolite.

2. The catalyst system of claim 1, wherein the metal of the at least one transition metal or transition metal sulfide in component (a) is a Group VI to Group X metal.

3-4. (canceled)

5. The catalyst system of claim 1, wherein the oxide-containing support having a low acidity in component (a) is silicon oxide (SiO₂), aluminum oxide (Al₂O₃), cerium (IV) oxide (CeO₂), zirconium dioxide (ZrO₂), titanium dioxide (TiO₂), or a mixture thereof.

6. The catalyst system of claim 1, wherein the acid zeolite in component (b) does not contain metal supported on the acid zeolite.

7. (canceled)

8. The catalyst system of claim 1, wherein the catalyst system is a physical mixture of component (a) and component (b), optionally wherein the weight ratio of component (a) to component (b) ranges from about 2:1 to about 1:2.

9-10. (canceled)

11. A process for chemically treating a carbon-containing feedstock, the process comprising:
contacting the carbon-containing feedstock with a hydrogen-containing stream in the presence of a catalyst system, the catalyst system comprising:
(a) at least one transition metal or transition metal sulfide supported on an oxide-containing support having a low acidity, and
(b) an acidic zeolite,
to produce an alkane-containing product stream.

12. (canceled)

13. A process for depolymerizing a polymer-based feedstock, comprising:
reacting a polymer-based feedstock with a hydrogen-containing stream in the presence of a catalyst system, the catalyst system comprising:
(a) at least one transition metal or transition metal sulfide supported on an oxide-containing support having a low acidity, and
(b) an acidic zeolite,
to depolymerize the polymer-based feedstock and form an alkane-containing product stream.

14. The process of claim 11, wherein the reaction is a one-step, hydrocracking reaction, optionally wherein the process is a one-pot process.

15. The process of claim 11, wherein the metal of the at least one transition metal or transition metal sulfide in component (a) is a Group VI to Group X metal.

16-17. (canceled)

18. The process of claim 11, wherein the oxide-containing support having a low acidity in component (a) is silicon oxide (SiO₂), aluminum oxide (Al₂O₃), cerium (IV) oxide (CeO₂), zirconium dioxide (ZrO₂), titanium dioxide (TiO₂), or a mixture thereof.

19. The process of claim 11, wherein the acid zeolite in component (b) does not contain metal supported on the acid zeolite.

20. (canceled)

21. The process of claim 11, wherein the catalyst system is a physical mixture of component (a) and component (b), optionally wherein the weight ratio of component (a) to component (b) ranges from about 2:1 to about 1:2.

22. The process of claim 11, wherein the carbon-containing feedstock is a polymer-based feedstock.

23-25. (canceled)

26. The process of claim 11, wherein the reaction is carried out at a temperature ranging from about 200 to about 500° C.

27. (canceled)

28. The process of claim 11, wherein the alkane-containing product stream comprises C1-C20 hydrocarbons, optionally wherein the process has a C1-C20 alkane product yield of at least 50%.

29. The process of claim 11, wherein the process has a selectivity towards a naphtha product of at least 50% by weight.

30. The process of claim 11, wherein the process produces less than 10% by weight of solid product stream, and/or wherein the process produces less than 10% by weight of diesel or kerosene product.

31. The process of claim 11, further comprising
separating the alkane-containing product stream into two or more different product streams based on the molecular weight of the components of the alkane-containing product stream.

32-33. (canceled)

34. An alkane-containing mixture obtained by the process of claim 11, optionally wherein the mixture comprises at least 50% by weight of naphtha product, based on the total weight of the mixture.

35. (canceled)

36. A method for selectively converting a polymer-based feedstock to a liquid naphtha product, comprising:
reacting a polymer-based feedstock with a hydrogen-containing stream in the presence of a catalyst system comprising:
(a) at least one transition metal or transition metal sulfide supported on an oxide-containing support having a low acidity, and
(b) an acidic zeolite,
to depolymerize the polymer-based feedstock and form a liquid naphtha product containing at least 50% by weight of C4-C12 hydrocarbons; less than 10% by weight of solid product stream; and optionally less than 10% by weight of diesel or kerosene product,
wherein the reaction temperature ranges from about 300-450° C. and hydrogen pressure ranges from about 5 to 100 bar.
